(19) United States
(12) Patent Application Publication
(10) Pub. No.: US 2002/0099317 A1
(43) Pub. Date: Jul. 25, 2002

(54) Mid-foot prosthetic device

(76) Inventor: David B. Plotkin, Short Hills, NJ (US)

Correspondence Address: STROOCK & STROOCK & LAVAN LLP, 180 Maiden Lane, New York, NY, 10038, US

(21) Appl. No.: 10/106,095

(22) Filed: Mar. 25, 2002

Related U.S. Application Data
(63) Continuation of application No. 09/621,786, filed on Jul. 21, 2000.
(60) Provisional application No. 60/144,922, filed on Jul. 21, 1999.

(51) Int. Cl. A61F 13/00; A61F 13/06

(52) U.S. Cl. 602/66

(57) ABSTRACT

A prosthetic support for reducing pronation of the mid-foot is provided. The support includes a sleeve to be pulled over the toes and positioned under the nervicular bone at the mid-foot position. A contoured resilient pad is held in position on the sleeve and positioned under the nervicular bone to medially support the mid-foot.

### CROSS-REFERENCE TO RELATED APPLICATIONS

This is a continuation of application Ser. No. 09/621,786, filed Jul. 21, 2000, now pending. application Ser. No. 09/621,786 in turn, claims priority of Provisional Application Serial No. 60,144,922, dated Jul. 21, 1999. The entire disclosure of application Ser. No. 09/621,786 is hereby incorporated herein by reference.

### BACKGROUND OF THE INVENTION

This invention is directed to a mid-foot prosthetic device and in particular to a prosthetic sleeve having a support pad that is positioned central to and under the nuvicular bone to support the mid-foot of a patient.

Heretofore the general public and in particular athletes have observed that the pronation of the mid-foot causes several injuries which are referred to as “overuse” injuries. Among these “overuse” injuries are: Plantar Fascititis; Tibial Tendinitis (anterior and posterior), Peroneal; heel spur syndrome; Tarsal pain; and shin splints. One way of providing relief for these injuries is to wrap tape around the medial mid-foot or strap the bottom of the foot. However, the problem with such taping or strapping is that it often will cause skin reactions, it is difficult to maintain a consistent amount of pressure and hence the tape has to be wound too tight. This problem of the tape loosening is particularly assacerbated during athletic activity which is a major cause of “overuse” injury. Accordingly, a mid-foot prosthetic device that can apply a force under the nervicular bone to support the mid-foot in a consistent and uniform manner is desired.

### SUMMARY OF THE INVENTION

Generally speaking in accordance with the invention, a prosthetic support for the medial mid-foot position is provided. The support includes an elastic sleeve adapted to be pulled over the toes and positioned under the nervicular bone. A contoured resilient pad is held in position on the elastic sleeve and in response to the elastic sleeve being pulled over the toe and positioned under the nervicular bone medially supports the mid-foot whereby over pronation is reduced.

Accordingly, the instant invention provides an improved prosthetic device that reduces pronation and alleviates pain caused by “overuse” injury.

Other objects and features of the present invention will become apparent from the following detailed description, considered in conjunction with the accompanying drawing figures. It is to be understood, however, that the drawings, which are not to scale, are designed solely for the purpose of illustration and not as a definition of the limits of the invention, for which reference should be made to the appended claims.

The invention accordingly comprises the features of construction, combination, of elements, and arrangement of parts which will be exemplified in the construction hereinafter set forth, and the scope of the invention will be indicated in the claims.

### DETAILED DESCRIPTION OF THE DRAWINGS

In the drawing figures, which are not to scale, and which are merely illustrative, and wherein like reference numerals depict like elements throughout the several views:

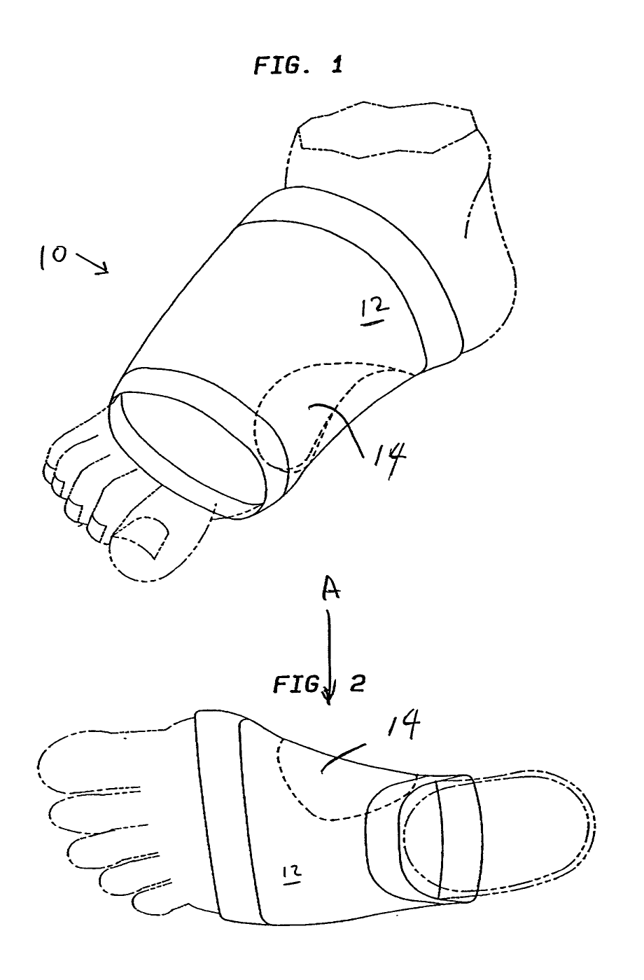

FIG. 1 is a perspective view of a mid-foot prosthetic sleeve device wrapped around a foot, in accordance with a preferred embodiment of the instant invention;

FIG. 2 is top plan view of the mid-foot prosthetic sleeve device depicted in FIG. 1;

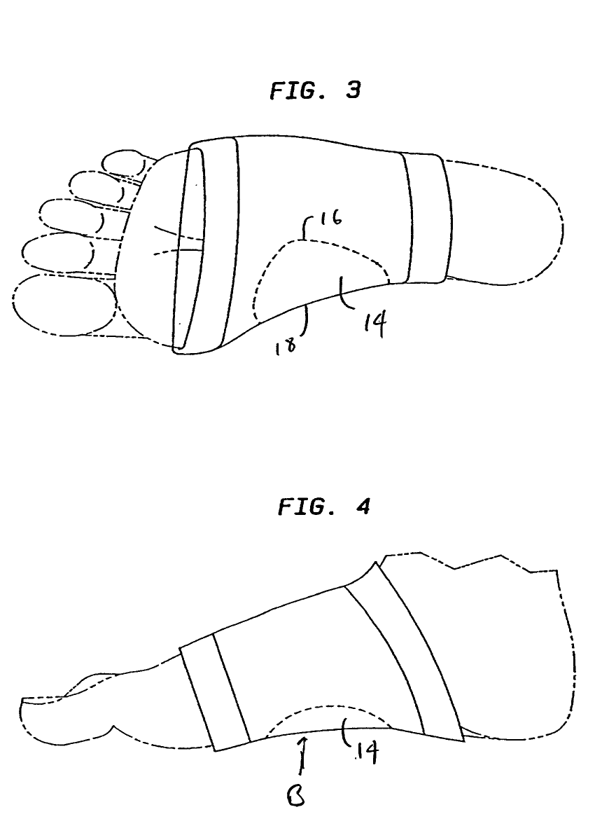

FIG. 3 is a bottom plan view of the mid-foot device depicted in FIG. 1;

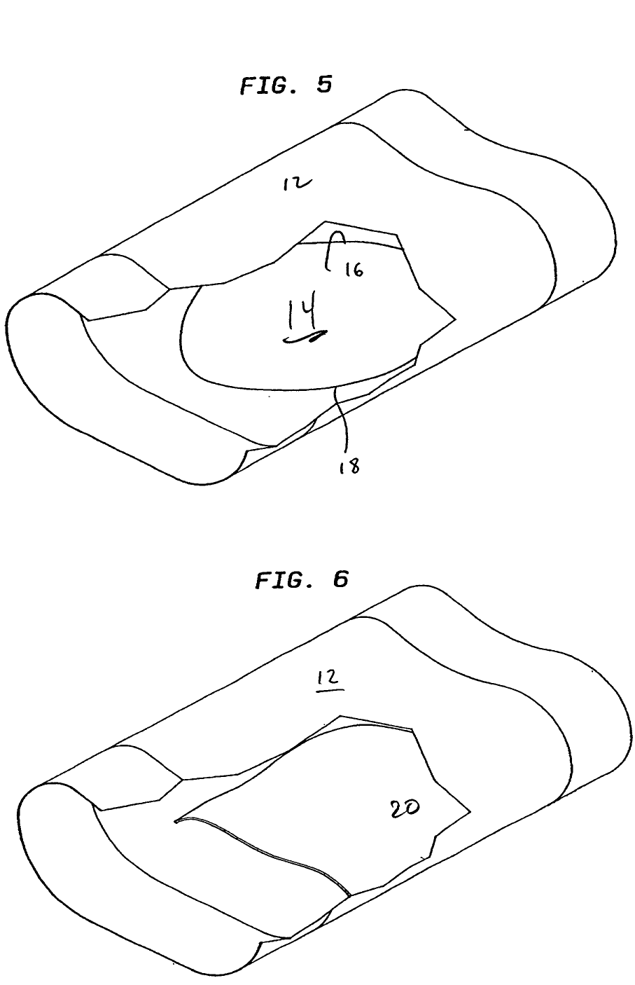

FIG. 4 is an elevational side view of the mid-foot prosthetic sleeve device depicted in FIG. 1;

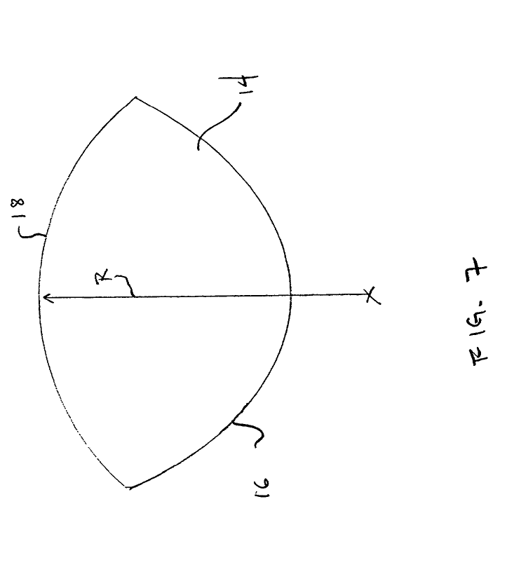

FIG. 5 is a cut away view of the mid-foot prosthetic sleeve, depicting a support pad in accordance with the instant invention;

FIG. 6 is a cut away view of the mid-foot prosthetic sleeve with a pocket for receiving and positioning the pad in accordance with an alternative embodiment of the instant invention;

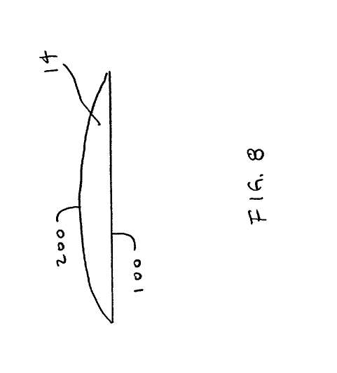

FIG. 7 is a plan view of a preferred embodiment of the support pad; and

FIG. 8 is a side view of the support pad of FIG. 7.

### DETAILED DESCRIPTION OF THE PREFERRED EMBODIMENTS

Generally speaking in accordance with this invention, reference is made to FIGS. 1-5 herein, a prosthetic device generally indicated at 10 includes a sleeve 12 made of an elastic material that is capable of applying certain pressure and to maintain a position of the pad at the mid-foot. Such materials are commonly used in ankle wraps, compression bandages and elastic support hosiery.

In an exemplary embodiment, a pad 14 is formed of a shape that contours to the medial arch of a person's foot. As illustrated in FIGS. 5 and 7, the shape of the pad 14 is defined by a smaller radiused periphery 16 intersecting a larger radiused periphery 18. Peripheries 16 and 18 may also be elliptical, hyperbolic, parabolic or ovoid, or any combination thereof, as a matter of design choice. Periphery 18 may also be substantially or completely straight. The pad is also preferably constructed of a firm yet resilient material, so as to conform to the shape of the bottom of the foot to which it will be applied while still providing the necessary support. Accordingly, the pad may be sponge or foam rubber, plastic, or any other of a wide variety of resilient, elastically deformable polymers, plastics and materials, or combinations thereof, as a matter of design choice within the level of skill of then routineer in the art. As seen in FIG. 8, the pad includes a generally flat bottom surface 100 and a generally convex top surface 200 preferably oriented to face toward the medial section of the foot. (FIG. 8). In a preferred embodiment of the invention, the pad can either be stitched to the elastic material in order to secure the pad with reference to the elastic material or it can be glued using an appropriate adhesive, or secured in any other art-recognized manner.

In an alternate embodiment depicted in FIG. 6, a pocket 20 can be attached to the elastic sleeve 12 so that the rubber pad 14 can be selectably inserted into and removed from the pocket. However, when utilizing a pocket, the pocket must be positioned so that that the pad 14 may be secured sufficiently in relationship to the elasticity to assure that the pad is position at the proper position under the nervicular bones to support the mid-foot.

In operation, the elastic sleeve 17 is slid over the toes to position the sleeve at the mid-foot position A of the patient. The final position B of the sleeve is such that the pad is positioned under the nervicular bone to restrict frontal plane movement.

Thus the present invention provides a support for the medial mid-foot position comprising an elastic sleeve adjusted to be pulled over the toes and positioned under the nervicular bone; and a contoured resilient pad constructed and arranged to be positioned under the nervicular bone to immediately support the mid-foot.

It will thus be seen that the objects set forth above, among those made apparent from the preceding description, are efficiently attained and, since certain changes may be made in the above construction(s) without departing from the spirit and scope of the invention, it is intended that all matter contained in the above description or shown in the accompanying drawing(s) shall be interpreted as illustrative and not in a limiting sense.

It is also to be understood that the following claims are intended to cover all of the generic and specific features of the invention herein described and all statements of the scope of the invention which, as a matter of language, might be said to fall therebetween.

Thus, while there have been shown and described and pointed out fundamental novel features of the invention as applied to preferred embodiments thereof, it will be understood that various omissions and substitutions and changes in the form and details of the disclosed invention may be made by those skilled in the art without departing from the spirit of the invention. It is the intention, therefore, to be limited only as indicated by the scope of the claims appended hereto.

What is claimed is:

1. A prosthetic support for a medial mid-foot position at the nevicular bone comprising a sleeve that surrounds the foot at the mid-foot position in a pad secured by the sleeve at the nevicular bone to medially support the mid-foot position.

2. A prosthetic support as claimed in claim 1, wherein said sleeve is elastic to permit said sleeve to be pulled over the toe and positioned securely about the mid-foot position.

3. A prosthetic support as claimed in claim 1, wherein said pad is a contoured resilient pad.

4. A prosthetic support as claimed in claim 3, wherein said sleeve is elastic.

5. A prosthetic support as claimed in claim 4, wherein said sleeve includes a pocket therein for permitting said pad to be insertable into and removable from said pocket.

6. A prosthetic support as claimed in claim 3, wherein the pad has an outer periphery substantially defined by a first radius periphery and by a second intersecting radius periphery.

7. A prosthetic support as claimed in claim 6, wherein the radius of said second radius periphery is greater than the radius of said first radius periphery.

8. A prosthetic support as claimed in claim 3, wherein said pad has a first substantially flat surface and a generally convex top surface, said top surface being adapted to be positioned at said nevicular bone.
